(19) United States
(12) Patent Application Publication
(10) Pub. No.: US 2003/0232016 A1
(43) Pub. Date: Dec. 18, 2003

(54) Nerve identification and sparing method

(76) Inventor: Russell Heinrich, Madison, CT (US)

Correspondence Address: Paul R. Audet, Senior Patent & Trademark Counsel, U.S. Surgical, a divison of Tyco, Healthcare Group LP, 150 Glover Avenue, Norwalk, CT, 06856, US

(21) Appl. No.: 10/418,353

(22) Filed: Apr. 17, 2003

Related U.S. Application Data
(60) Provisional application No. 60/373,691, filed on Apr. 17, 2002.

(51) Int. Cl. A61K 49/00; A61N 1/30

(52) U.S. Cl. 424/9.6; 604/20

(57) ABSTRACT

A method for identifying and sparing nerves during a surgical procedure is disclosed wherein a particular indicator is applied to a surgical site to identify nerve tissue. The indicator visually distinguishes the nerves from surrounding tissue. The identified nerves are then spared during the surgical procedure.

### CROSS REFERENCE TO RELATED APPLICATIONS

This application claims priority from U.S. Provisional Application Serial No. 60/373,691 filed on Apr. 17, 2002, the entire contents of which are incorporated herein by reference.

### BACKGROUND

1. Technical Field

The present disclosure relates to a method for locating and identifying a nerve and, more particularly to a method for locating and identifying the nerves surrounding the prostate gland during a radical prostatectomy.

2. Background of Related Art

The locating and identifying of nerves surrounding an area of tissue has grown in importance over the last several years as surgeons attempt to avoid severing or damaging a nerve during the sectioning or excision of surrounding tissue. For example, during surgical procedures, the operating surgeon must avoid cutting or injuring nerves that are essential for specific motor and sensory functions. To avoid cutting or injuring such nerves, the surgeon may use a nerve stimulator to determine the exact location of a nerve to guard against inadvertent cutting.

An example of a nerve stimulator used for this purpose is disclosed in U.S. Pat. No. 2,704,064 to Fizzell et al. that relates to a neuromuscular stimulator having two probes for passing a current to a subcutaneous nerve. The probes are placed on the body in the area of the nerve to be stimulated and an operating surgeon watches for a response to the applied current. If a response to the current is observed, the surgeon avoids cutting in that particular area to prevent inadvertent severing or damaging of a nerve. In effect, by probing carefully, the surgeon, for example, may excise tumorous tissue without destroying nerves affecting specific body functions.

Localization of a nerve by conventional electro-stimulation is further complicated if the nerve comprises multiple, microscopic branches or if the nerve is disposed in a region of the body difficult to reach because of the surrounding anatomy. Still other nerves, such as autonomic nerves, can be difficult to locate because such nerves evoke response patterns that cannot be immediately observed or interpreted by the operating physician administering the electro-stimulus to the tissue site.

For example, stimulation of the cavernosal nerve evokes a multi-stage response comprising: (1) relaxation of the smooth muscles of the arterioles supplying the penis; (2) dilation of the arteries leading to the penis; (3) constriction of the veins carrying blood away from the penis; and (4) accumulation of blood in the cavernosa. Such a multi-stage response is especially difficult to interpret because the response might not occur until some time after application of the stimulus. In some instances, the delay may be as long as two seconds or more. Given the aforementioned factors, observation of the response pattern alone may not lead to a determination of the location of a nerve.

These concerns are particularly pertinent in radical prostatectomy procedures. During a radical prostatectomy procedure the nerves surrounding the prostate gland, which nerves control urinary continence and erectile function, are exposed and accordingly, during the prostatectomy procedure, every effort is made to spare these nerves.

In endoscopic systems, it is well known to irradiate an internal organ with visible radiation to obtain a visible image and then to apply to the organ a fluorescent dye that concentrates in tumors over a period of time. The dye is allowed to concentrate, and then the internal organ is irradiated with excitation radiation for the dye to obtain a second fluorescent image. A body part having abnormal or diseased tissue, such as a cancer, may be identified by comparing and image produced by visible radiation of the internal organ with the image produced by fluorescence. To aid in visualizing the images received, endoscopic systems commonly utilize a television camera attached to a fiber optic scope having an optical guide fiber for guiding a beam from an external radiation source to the internal organ, and another optical guide fiber for transmitting a fluorescent image of the affected area to a television monitor for viewing.

An example of such a practice is disclosed in U.S. Pat. No. 6,299,860 to Luiken. Luiken relates generally to a method for using fluorescent indicating substances that target and bind with diseased body tissue. According to the teachings of Luiken, diseased tissue retains the fluorescent indicator thereby allowing the observer to distinguish between healthy and diseased tissue regions in a patient's body.

Accordingly, a need exists for a method of identifying nerves during surgical procedures and, more particularly to a method for identifying and locating nerves during a radical prostatectomy procedure so that these nerves can be spared. In addition, a need exists for a method of identifying nerves during a surgical procedure using surgical dyes and/or radiopharmaceuticals that distinguishes the nerve from the surrounding tissue.

### SUMMARY

A nerve identification and sparing method for use on a body of a patient when performing a surgical procedure is herein disclosed. The nerve identification and sparing method, according to the present disclosure, includes the steps of incising a patient's body to expose a target area; adding an indicator to the target area; allowing the indicator to accumulate in nerves of the patient at the target area; viewing the nerves containing the indicator; and performing surgery on body tissue at the target area by avoiding the nerves containing the indicator.

It is preferred that a biocompatible fluorescent indicator is used to identify the nerves in the patient's body. Alternately, it is envisioned that radiopharmaceuticals or other radioactive substances that accumulate in nerve tissue may be used to identify nerves according to the method herein disclosed.

It is an object of the present disclosure to provide a nerve identification and sparing method overcoming the drawbacks of the prior art nerve identification methods.

It is another object of the present disclosure to provide a nerve identification and sparing method that allows for visualization of nerves that are very small and difficult to see using current methods.

It is still a further object of the present disclosure to provide a nerve identification and sparing method that enables an operating surgeon to spare and avoid severing or injuring nerves during a radical prostatectomy procedure. These objects, together with other objects of the disclosure, along with various features of novelty that characterize the disclosure, are pointed out with particularity in the claims annexed hereto and forming a part of this disclosure.

### BRIEF DESCRIPTION OF THE DRAWINGS

The accompanying drawings, which are incorporated in and constitute a part of this specification, illustrate embodiments of the present disclosure and, together with a general description of the present disclosure given above, and the detailed description of the embodiments given below, serve to explain the principles of the present disclosure.

FIG. 1 is a schematic representation of a prostatectomy surgical site prior to performance of the nerve identification and sparing method in accordance with the present disclosure; and

FIG. 2 is a schematic representation of a prostatectomy surgical site after the performance of the nerve identification and sparing method in accordance with the present disclosure.

### BRIEF DESCRIPTION OF PREFERRED EMBODIMENTS

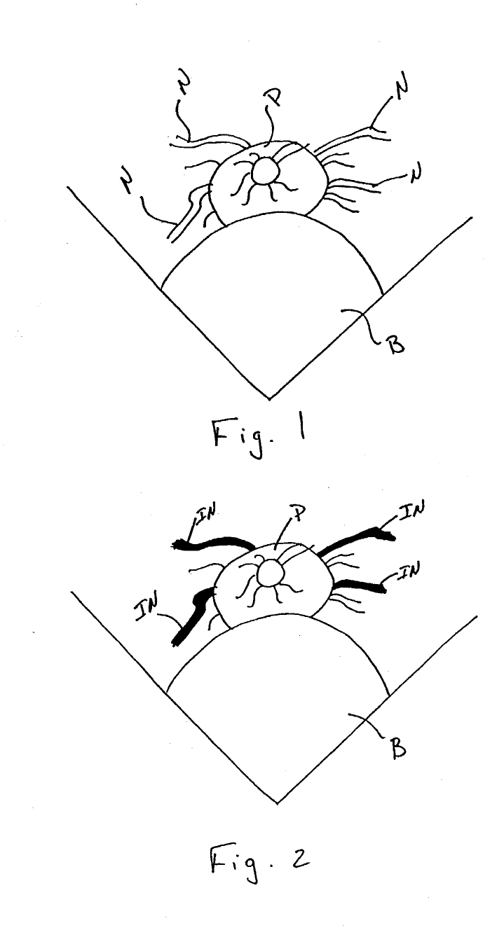

Preferred embodiments of the presently disclosed nerve identification and sparing method will now be described in detail with reference to the drawing figures wherein like reference characters identify similar or identical elements. Referring now in detail to FIG. 1, a nerve identification and sparing method in accordance with the present disclosure is illustrated. Although the method according to the present disclosure offers significant advantages to a radical prostatectomy procedure, it will be understood that the device is applicable for use in any procedure in which the identification and sparing of nerves is desired.

In general, there are primarily two types of radical prostatectomy approaches for the removal of prostate cancer, the retropubic approach, and the perineal approach. In the retropubic approach, a long up-and-down incision is made in the midline of the abdomen from the navel to the pubic bone. After the lymph nodes have been removed for study by the pathologist and a determination has been made to proceed with the removal of the prostate gland, the space underneath the pubic bone is cleaned and dissected and the removal of the entire prostate gland is generally begun at the end that is farthest from the bladder, next to the external urethral sphincter.

The prostatic urethra is divided at this point. First, the prostatic urethra and the prostate gland through which it goes is pulled upwards toward the bladder while the dissection continues behind the prostate gland, separating it from the layer of tissue that is connected to the rectum on its other side. As the dissection continues between the prostate and the rectum, the seminal vesicles, which are behind the base of the bladder, will be removed along with the prostate gland. Once the seminal vesicles are free, the entire prostate gland and the seminal vesicles are removed. The bladder neck is then stitched closed to a small enough diameter so that it is about the same size as the stump of the urethra from which the prostate was detached. The bladder neck is then pulled down into the pelvis, snuggled up against the urethral stump, and stitched thereto. This stitching is done typically around a Foley catheter, which has been inserted through the penis all the way into the bladder.

In the perineal approach, an inverted “U” shaped incision is made going right over the anus, with the center of the “U” about three centimeters above the margin of the anus. The prostate gland is then freed from its surrounding structures by gentle dissection, and the urethra at the end of the prostate farthest from the bladder is isolated and divided. The bladder neck is freed from the prostate, and, once the prostate gland has been removed and the bladder neck has been closed sufficiently so that the size of its opening approximates the size of the urethral opening, the urethra and the bladder neck are stitched together. Once again, a Foley catheter is left in place postoperatively for about two weeks.

In accordance with the present disclosure, the nerve identification and sparing method as applied to a radical prostatectomy procedure (i.e. either a retropubic or a perineal approach) will now be described. Initially, as seen in FIG. 1, the operating surgeon creates an incision, either retropubically or perineally, in the patient that exposes the prostate “P” and the bladder “B.” Next, an indicator is introduced to the region surrounding the nerves “N” that are in the vicinity of prostate “P” and bladder “B” by the operating surgeon. It is contemplated that the indicator is introduced using standard methods including, but not limited to, injecting, swabbing, pouring, and inserting. A number of indicators are available for identifying nerves in surrounding tissue.

Preferably, the indicator is a lipophilic tracer belonging to either the dialkylcarbocyanine family of dyes or the dialkylaminostyryl family of dyes. Advantageously, the indicator is 1,1′-dilinoleyl-3,3,3′,3′-tetramethylindocarbocyanine perchlorate (oil) or 1,1′-dilinoleyl-3,3,3′,3′-tetramethylindocarbocyanine, 4-chlorobezenesulfonate (solid) that is commercially available as FAST DiI™ from Molecular Probes, Inc. It is packaged either as sticky oil or in solid form and is warmed to approximately 40° C. to facilitate transport of the dye. FAST DiI™, is introduced to the surgical site in a conventional manner, as discussed above.

The indicator is allowed to accumulate or concentrate in the nerves “N” for a period of time. In so doing, the nerves “N” accumulate enough of the indicator so that when the nerves “N” are exposed or excited with a particular wavelength, or range of wavelengths, of radiation, the nerves “N” containing the indicator will fluoresce, as depicted by the reference character “IN” in FIG. 2, and be either detectable with the naked eye, through a filter or with the aid of probe (i.e., gamma type, endoscope, camera with a fiber optic scope, etc.).

Advantageously, the indicator has a higher affinity for nerve tissue as compared to the tissue surrounding the nerves at the surgical site. Therefore, the preferred indicator will substantially accumulate or concentrate in nerves “N” rather than in the surrounding tissue providing a high degree of accuracy for identifying nerves “N” rather than adjacent tissues.

In the preferred embodiment, where a fluorescent indicator, such as FAST DiI™, is used, an operator irradiates the target surgical site containing the indicator with radiation at the absorption wavelength of the fluorophore. FAST DiI™ has an absorption wavelength of 549 nanometers; therefore, it is desirable to provide a source of radiation wherein the output is concentrated in the range between 510 nanometers and 560 nanometers. A suitable filter placed between the radiation source and the surgical site will substantially prevent wavelengths outside this range from being transmitted to the surgical site. The radiation source can be any known source of visible light capable of illuminating the surgical site.

In the preferred method, the irradiated nerves “IN” are identified using a filter placed in the operating surgeon's view of the surgical site. FAST DiI™ fluoresces best at 565 nanometers. A suitable filter for identifying the irradiated nerves “IN” has maximum transmission for wavelengths in the range between 560 nanometers and 650 nanometers thereby allowing the operating surgeon to distinguish the irradiated nerves “IN” from the surrounding tissue.

It is contemplated that, if the dye has a short half-life, the dye can be injected into the target area in order to act as a predictor of urologic sexual function after the radical prostatectomy has been performed.

With the nerves “N” irradiated and/or otherwise visible, when performing the radical prostatectomy, the operating surgeon can more easily avoid severing the irradiated nerves “IN” when excising the prostate “P” from the target area. Accordingly, the nerve identification and sparing method according to the present disclosure would allow visualization of the nerves “N” surrounding the prostate “P,” which nerves “N” are very small and difficult to see with the naked eye or with current surgical techniques.

While the above-disclosed method has been described in connection with a radical prostatectomy, it is envisioned that similar methods can be performed with regard to various other surgical procedures. It will be understood that various modifications may be made to the embodiments of the presently disclosed nerve identification and sparing method disclosed herein. Therefore, the above description should not be construed as limiting, but merely as exemplifications of preferred embodiments. Those skilled in the art will envision other modifications within the scope and spirit of the present disclosure.

What is claimed is:

1. A nerve identification and sparing method, comprising the steps of:
incising tissue of a patient to expose a surgical site having a plurality of nerves;
adding an indicator that is absorbable by the plurality of nerves to the surgical site;
allowing the indicator to accumulate in the nerves of the patient at the surgical site;
identifying the nerves containing the indicator; and
performing a surgical procedure at the surgical site by avoiding the nerves identified as containing the indicator.

2. The nerve identification and sparing method of claim 1, wherein the indictor is a fluorescent dye excitable by radiation.

3. The nerve identification and sparing method of claim 2, wherein the indicator is lipophilic tracer.

4. The nerve identification and sparing method of claim 3, wherein the adding step includes the indicator being selectively absorbable by the plurality of nerves

5. The nerve identification and sparing method of claim 4, wherein the identifying step includes irradiating the nerve containing the indicator.

6. The nerve identification and sparing method of claim 5, further comprising the step of:
viewing the irradiated nerve containing the indicator through a filter.
